(19) United States
(12) Patent Application Publication
(10) Pub. No.: US 2023/0143549 A1
(43) Pub. Date: May 11, 2023

(54) OPTOMETRY SYSTEM AND NON-TRANSITORY COMPUTER-READABLE STORAGE MEDIUM

(71) Applicant: NIDEK CO., LTD, Gamagori, Aichi (JP)

(72) Inventors: Arisa SHIMAZAKI, Gamagor-shii, Aichi (JP); Taeko HORINO, Gamagori-shi, Aichi (JP); Yukito HIRAYAMA, Gamagori-shi, Aichi (JP); Michihiro TAKII, Gamagori-shi, Aichi (JP); Jouji SEKO, Gamagori-shi, Aichi (JP)

(73) Assignee: NIDEK CO., LTD, Gamagori, Aichi (JP)

(21) Appl. No.: 17/915,356

(22) PCT Filed: Mar. 25, 2021
(86) PCT No.: PCT/JP2021/012742

§ 371 Date: Sep. 28, 2022

(30) Foreign Application Priority Data
Mar. 31, 2020 (JP) 2020-063526
Dec. 1, 2020 (JP) 2020-199931

(51) Int. Cl. A61B 3/032; A61B 3/00

(52) U.S. Cl. CPC A61B 3/032; A61B 3/0033

(57) ABSTRACT

This optometry system, for presenting a test chart to an eye being tested and subjectively measuring an optical characteristic of the eye being tested, is provided with a setting means which sets a reference value based on the subject's reaction time to a test chart, a reaction input means which inputs the response of the subject reading the test chart, a control means which automatically advances the test on the basis of an input signal from the response input means, and a guidance information output means which, on the basis of the reference value set by the testing means, outputs guidance information for guiding the subject to input a response during testing of the eye being tested. Hereby, it is possible to accurately carry out a subjective test on the eye being tested.

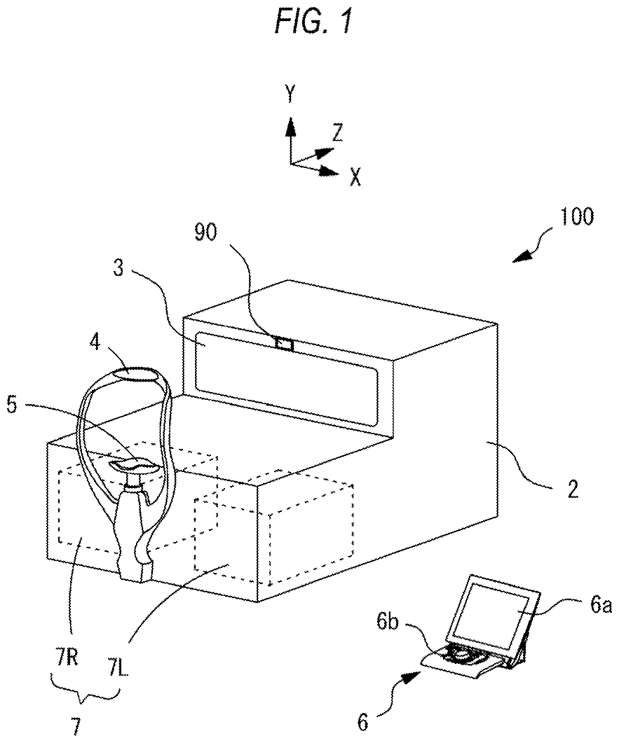

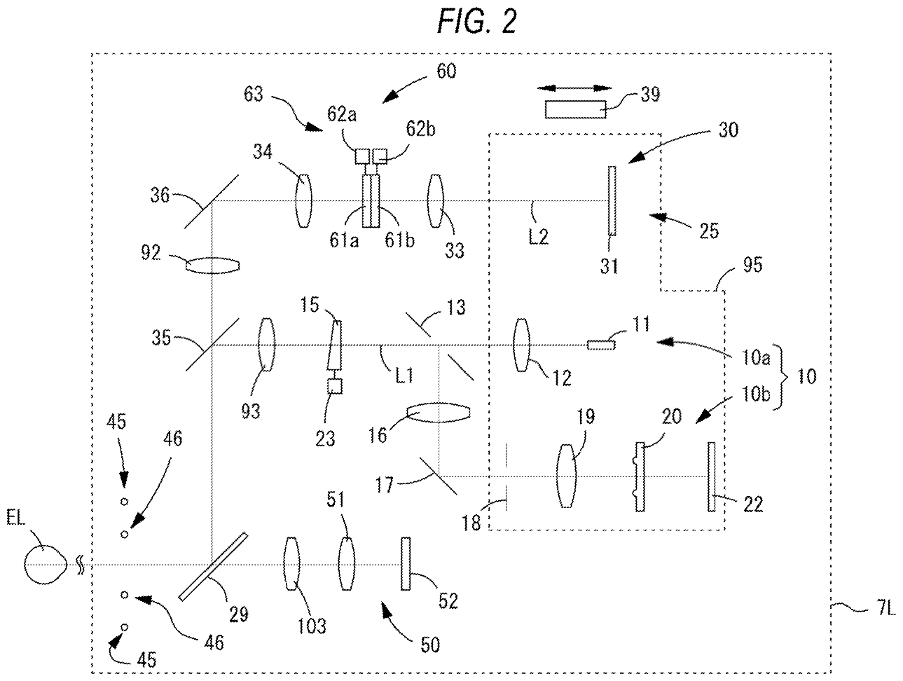

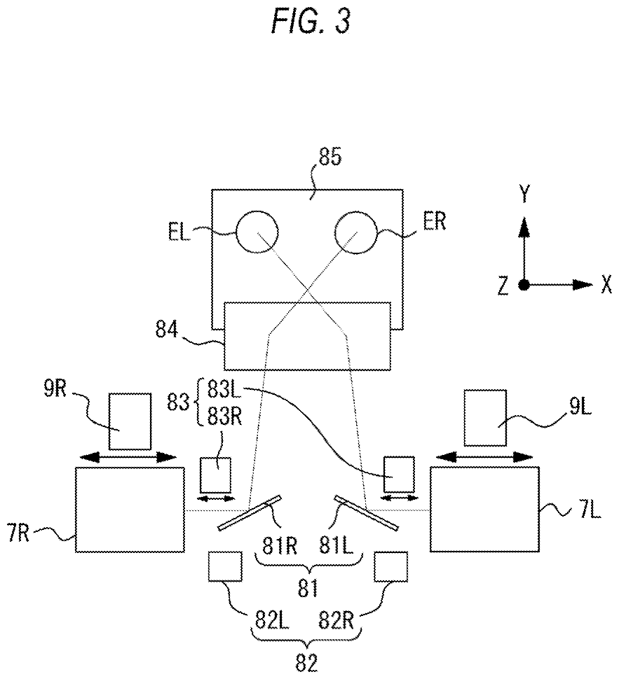

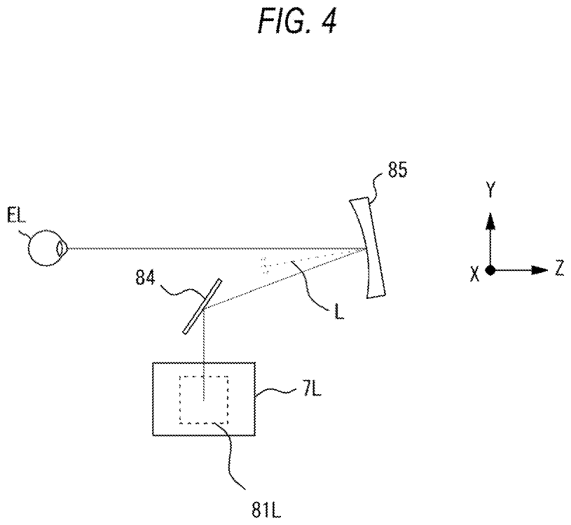

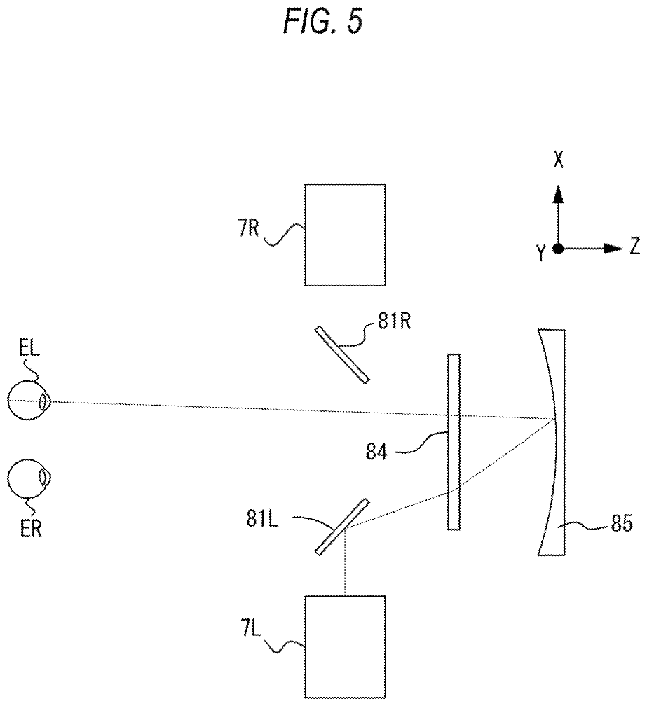

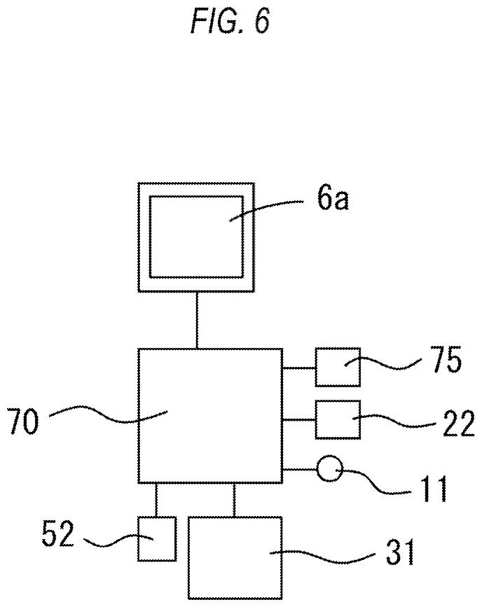

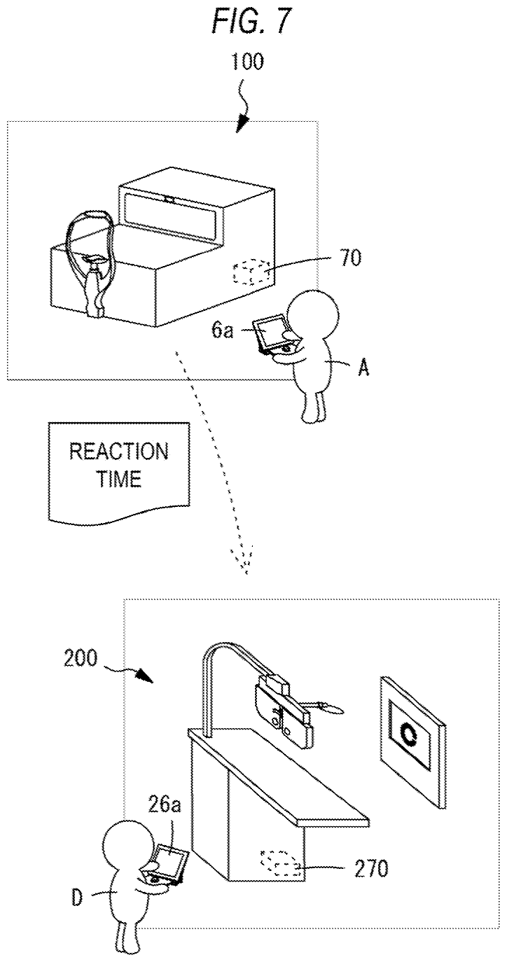

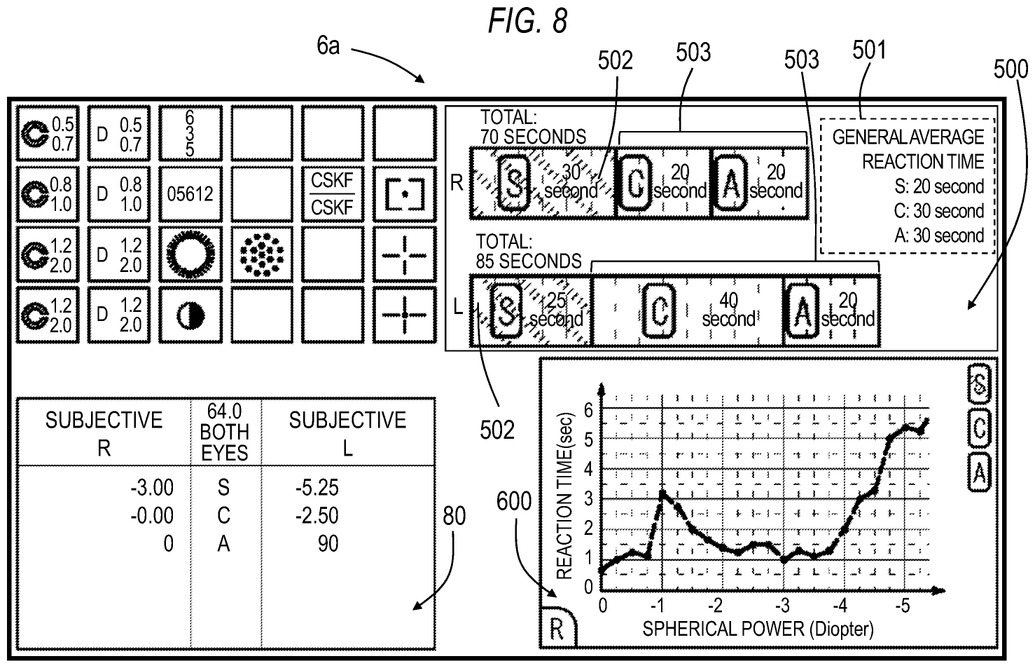

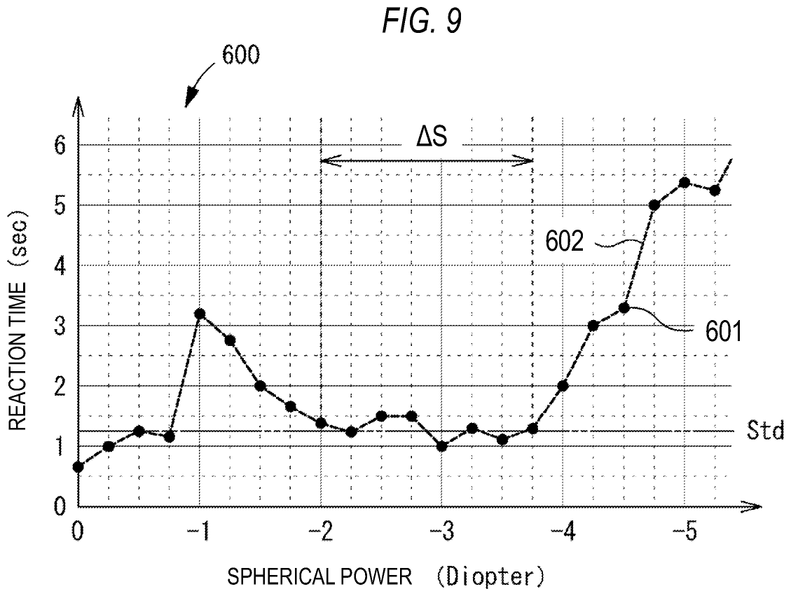

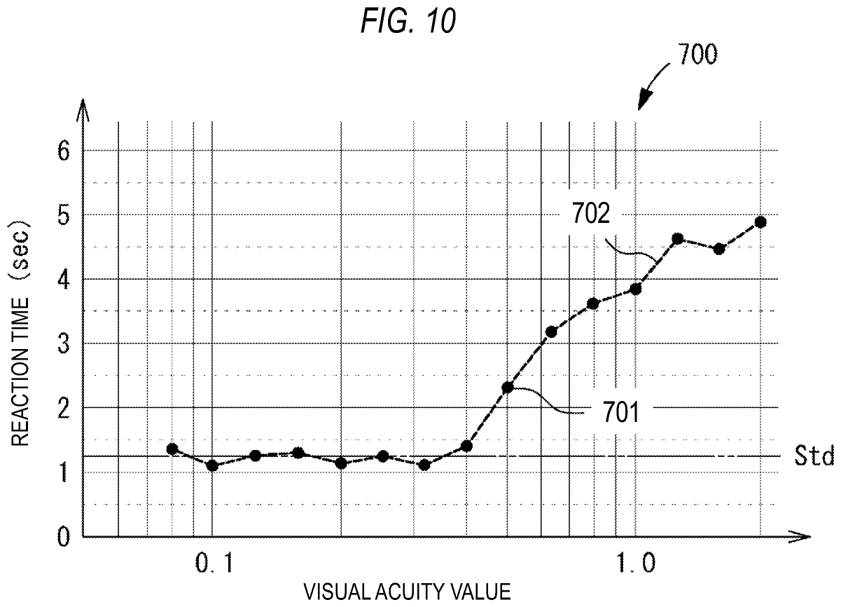

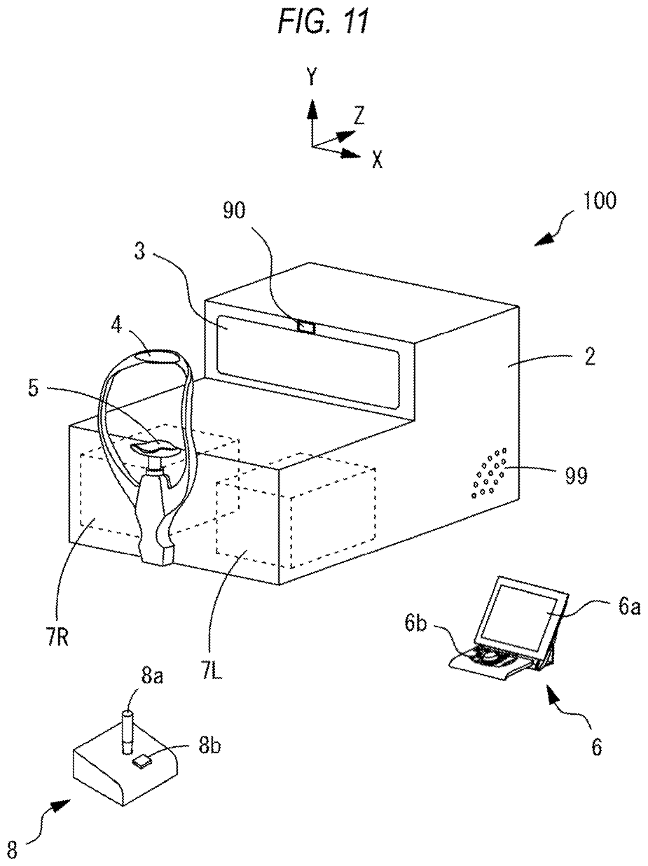

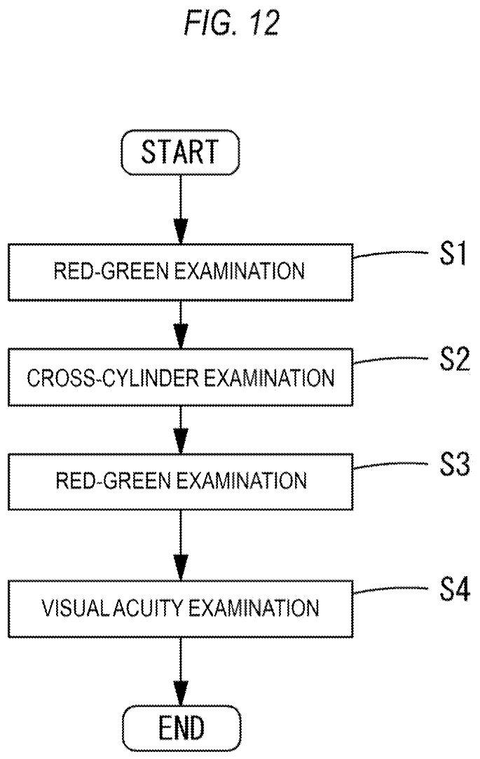

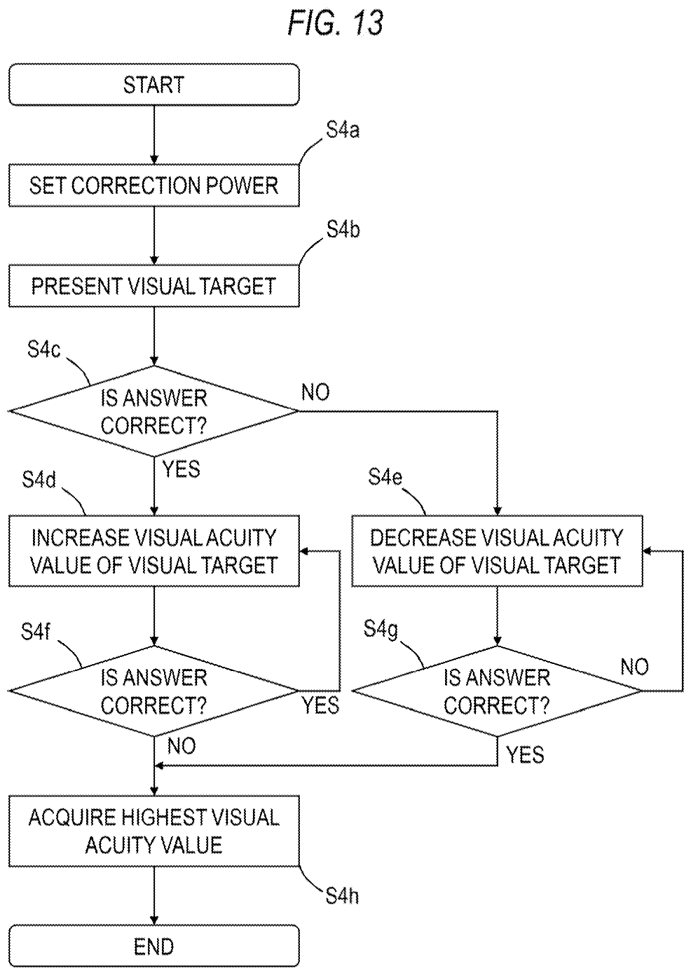

### TECHNICAL FIELD

The present disclosure relates to an optometry system and a non-transitory computer-readable storage medium storing an optometry program for subjectively measuring an optical characteristic of a subject eye.

According to a first aspect of the present disclosure, there is provided an optometry system for subjectively measuring an optical characteristic of a subject eye by presenting an examination visual target to the subject eye, the optometry system including: a response input unit by which an examinee reading the examination visual target inputs an answer; and a controller configured to: set a reference value based on a reaction time of the examinee with respect to the examination visual target; automatically proceed with an examination, based on an input signal from the response input unit; and output leading information, during the examination of the subject eye, for leading the examinee to input the answer, based on the set reference value.

According to the first aspect of the present disclosure, there is provided a non-transitory computer-readable recording medium storing an optometry program used in an optometry system for subjectively measuring an optical characteristic of a subject eye by presenting an examination visual target to the subject eye, the optometry program being executed by a processor to cause the optometry system to perform: a setting processing of setting a reference value based on a reaction time of an examinee with respect to the examination visual target; an answering processing in which the examinee reading the examination visual target inputs an answer; a control processing of automatically proceeding with an examination, based on an input signal of the answering processing; and a leading information output processing of outputting leading information, during the examination of the subject eye, for leading the examinee to input the answer, based on the reference value set in the setting processing.

According to a second aspect of the present disclosure, there is provided an optometry system for subjectively measuring an optical characteristic of a subject eye, the optometry system including: a visual target presenting unit that emits a target light flux toward the subject eye; a correction unit that changes an optical characteristic of the target light flux; and a controller configured to: control an operation of the optometry system; acquire a reaction time; and output the acquired reaction time, in which the reaction time is a time acquired based on a start timing at which measurement of the subject eye is started and a response timing after the start timing, the reaction time being a time required from a start of the measurement of the subject eye to a response with respect to the measurement of the subject eye, the start timing is a timing at which the visual target presenting unit or the correction unit is controlled by the controller, and the response timing is a timing at which a response signal with respect to the measurement is input by a response input unit.

The optometry system in the present embodiment may include at least a subjective optometry device (for example, an optometry device 100). For example, the subjective optometry device subjectively measures the optical characteristic of the subject eye by projecting the target light flux toward the subject eye and changing the optical characteristic of the target light flux. For example, the subjective optometry device may have a visual target presenting unit which will be described later, a correction unit which will be described later, and the like. Further, for example, the subjective optometry device may have a response input unit that will be described later.

### Visual Target Presenting Unit

The optometry system in the present embodiment includes a visual target presenting unit. The visual target presenting unit emits a target light flux toward the subject eye.

For example, the visual target presenting unit may be a display (for example, a display 31). Further, for example, a light source and a digital micromirror device (DMD) can be used as the visual target presenting unit. Further, for example, the visual target presenting unit may be a light source and a visual target plate.

For example, the target light flux from the visual target presenting unit may be emitted directly toward the subject eye. Further, for example, the target light flux from the visual target presenting unit may be indirectly guided toward the subject eye via a light projecting optical system (for example, a light projecting optical system 30). For example, the light projecting optical system may have at least one optical member for passing the target light flux emitted from the visual target presenting unit. As an example, a lens, a mirror, and the like may be provided.

The optometry system may include a visual target presenting unit as a member that partially configures the subjective optometry device. In this case, the subjective optometry device may be provided with the visual target presenting unit. In addition, the optometry system may include a visual target presenting unit as a visual target presentation device in addition to the subjective optometry device.

### Correction Unit

The optometry system in the present embodiment includes a correction unit. For example, the correction unit may include a correction optical system (for example, a correction optical system 60). For example, the correction optical system is arranged in the optical path of the light projecting optical system and changes the optical characteristic of the target light flux.

Further, as an example, the correction optical system may optically change the presentation position (presentation distance) of the visual target with respect to the subject eye to change the optical characteristic of the target light flux. In this case, the visual target presenting unit may be moved in the optical axis direction, or an optical element (for example, a spherical lens or the like) arranged in the optical path may be moved in the optical axis direction.

Further, for example, the correction optical system may arrange an optical element between the visual target presenting unit and the optical member for guiding the target light flux from the light projecting optical system toward the subject eye and control the optical element to, change the optical characteristic of the target light flux. In other words, the correction optical system may be a phantom lens refractometer (phantom correction optical system).

### Response Input Unit

The optometry system in the present embodiment includes the response input unit. The response input unit is a means for the examinee to input the answer obtained by reading the examination visual target. For example, the response input unit may be an operation means such as a lever switch, or a push button switch (for example, an examinee controller 8). Further, the response input unit may be a voice input means such as a microphone. In addition, the response input unit may be a detection means that detects movement of the line of sight or gestures of the examinee.

### Reaction Time Acquisition Means

The optometry system according to the present embodiment includes a reaction time acquisition means (for example, a controller unit 70). The reaction time acquisition means acquires a reaction time, in which the reaction time is a time acquired based on a start timing at which measurement of the subject eye is started and a response timing after the start timing, and the reaction time is a time required from a start of the measurement of the subject eye to a response with respect to the measurement of the subject eye, in which the start timing is a timing at which the visual target presenting unit or the correction unit is controlled by the controller, and the response timing is a timing at which a response signal with respect to the measurement is input by the response input unit (for example, a controller 6 and the subject controller 8).

For example, the start timing at which the measurement of the subject eye is started may be the timing at which a start signal serving as a trigger for controlling the visual target presenting unit or the correction unit is acquired. For example, the start timing may be the timing at which the start signal is automatically input based on an optometry program or the like. Alternatively, for example, the start timing may be the timing at which the start signal is input by the examiner's operation of the operation means (for example, the controller 6). Alternatively, for example, the start timing may be the timing at which the start signal input by the examiner's operation of the operation means is received.

For example, such a start timing may be the timing at which measurement is started individually in the process of measuring the examination item for the subject eye. More specifically, the start timing may be the timing at which either the visual target presenting unit or the correction unit is sequentially controlled in the process of measuring the examination item. For example, the start timing may be each timing at which a start signal for switching the visual target is input or received or each timing at which the visual target is switched based on the start signal in the process of measuring the examination item. The timing at which the visual target is switched may be the timing at which the visual acuity value of the visual target is switched or the timing at which the type of the visual target is switched. In addition, for example, the start timing may be each timing at which a start signal for switching the correction power for correcting the subject eye is input or received or each timing at which the correction power is switched based on the start signal in the process of measuring the examination item.

Further, for example, such a start timing may be the timing at which the measurement of the examination items for the subject eye is started. More specifically, the start timing may be the timing at which either the visual target presenting unit or the correction unit is first controlled in order to start measuring the examination item. For example, the start timing may be the timing at which a start signal for switching the visual target is first input or received, in which the start timing may be the timing at which the visual target is first switched based on the start signal in the measurement of the examination items. Further, for example, the start timing may be the timing at which a start signal for switching the correction power is first input or received, in which the start timing may be the timing at which the correction power is first switched based on the start signal in the measurement of the examination items.

For example, the response timing at which the response signal for the measurement is input may be the timing at which the response signal is acquired based on the operation of the response input unit. For example, the response timing may be the timing at which the response signal is input by operating the response input unit. Further, for example, the response timing may be the timing at which the response signal input by operating the response input unit is received. The response input unit may be operated by the examiner or may be operated by the examinee. In a case where the examiner operates the response input unit, the response input unit may also serve as the operation means.

For example, such a response timing may be the timing at which each measurement ends individually in the process of measuring the examination item for the subject eye. More specifically, the response timing may be each timing at which either the visual target presenting unit or the correction unit is sequentially controlled and the response signal with respect to this is input in the process of measuring the examination item. For example, the response timing may be each timing at which a response signal is input in accordance with the switching of the visual target, in which the response timing may be each timing at which the response of the examinee is input in the process of measuring the examination item. Further, for example, the response timing may be each timing at which a response signal is input in accordance with the switching of the correction power, in which the response timing may be each timing at which the response of the examinee is input in the process of measuring the examination item.

Further, for example, the response timing at which the response signal for the measurement of the subject eye is input may be the timing at which the measurement of the examination items for the subject eye is ended. More specifically, the response timing may be the timing at which either the visual target presenting unit or the correction unit is last controlled and the response signal with respect to this is input, in which the response timing may be the timing at which the response of the examinee is input in the measurement of the examination items.

As an example, the reaction time acquisition means may acquire each reaction time when sequentially switching at least one of the visual target or the correction power by starting time measurement at the start timing at which either the visual target presenting unit or the correction unit is controlled and ending time measurement at the response timing at which the response signal for this is input, in the process of measuring the examination item. That is, the reaction time may be acquired in real time during the examination.

Further, as an example, the reaction time acquisition means may acquire the entire response time required for the measurement of the examination items by starting time measurement at the start timing at which either the visual target presenting unit or the correction unit is first controlled and ending time measurement at the response timing at which the response signal is input in a case where either the visual target presenting unit or the correction unit is last controlled in the measurement of the examination items.

As an example, the reaction time acquisition means may acquire each response time when sequentially switching at least one of the visual target and the correction power from the difference between the start timing at which either the visual target presenting unit or the correction unit is controlled and the response timing at which the response signal for this is input, in the process of measuring the examination item.

Further, as an example, the reaction time acquisition means may acquire the entire reaction time required for the measurement of the examination items from the difference between the start timing at which either the visual target presenting unit or the correction unit is first controlled and the response timing at which the response signal is input in a case where either the visual target presenting unit or the correction unit is last controlled, in the measurement of the examination items.

For example, the reaction time acquisition means may acquire the first reaction time and the second reaction time at different start timings An example may be two or more reaction times in the process of measuring the examination item. In addition, an example may be the reaction time including the entire reaction time required for the measurement of the examination items and the reaction time excluding the start timing at which either the visual target presenting unit or the correction unit is first controlled.

Further, for example, the reaction time acquisition means may acquire the first reaction time and the second reaction time at different response timings. An example may be two or more reaction times in the process of measuring the examination item. In addition, an example may be the reaction time including the entire reaction time required for the measurement of the examination items and the reaction time excluding the response timing at which either the visual target presenting unit or the correction unit is last controlled.

Further, for example, the reaction time acquisition means may acquire the first reaction time and the second reaction time at different start timings and different response timings. An example may be two or more reaction times in the process of measuring the examination item. In addition, an example may be the reaction time including the entire reaction time required for the measurement of the examination items and the reaction time excluding the start timing at which either the visual target presenting unit or the correction unit is first controlled and the response timing at which either the visual target presenting unit or the correction unit is last controlled.

It is needless to say that, for example, the reaction time acquisition means may acquire a time different from the reaction time required from the start of measurement to the response. As an example, the reaction time acquisition means may acquire the examination time required for a series of measurements in the examination item. In this case, the reaction time acquisition means may acquire the examination time by adding the operation time for the operator to operate the response input unit to the entire reaction time required for the measurement of the examination item. More specifically, for example, the examination time may be acquired by adding the time from each response timing to the next start timing in the process of measuring the examination item to the entire reaction time required for the measurement of the examination item.

### Setting Means

The optometry system according to the present embodiment includes a setting means (for example, the controller 70). The setting means sets a reference value based on the reaction time of the examinee with respect to the examination visual target. In other words, a reference value as a reference for the time required for the examinee to respond using the response input unit after visually recognizing the examination visual target (that is, reaction time), is set. For example, by the setting means, it is possible to change the reference value to a different reference value for each examinee in consideration of individual differences in reaction speed of the examinee.

### State Acquisition Means

The optometry system according to the present embodiment includes a state acquisition means (for example, the controller 70). The state acquisition means acquires the state of the optometry system in a case where the visual target presenting unit or the correction unit is controlled by the control means which will be described later.

For example, the state acquisition means may acquire the state of the visual target presenting unit in a case where the visual target presenting unit is controlled by the control means. Further, for example, the state acquisition means may acquire the state of the correction unit in a case where the visual target presenting unit is controlled by the control means. Further, for example, the state acquisition means may acquire the state of the visual target presenting unit in a case where the correction unit is controlled by the control means. Further, for example, the state acquisition means may acquire the state of the correction unit in a case where the correction unit is controlled by the control means which will be described later. It is needless to say that the state which combined these may be acquired.

For example, the state of the visual target presenting unit may include at least one of the state of the type of the visual target, the state of the visual acuity value of the visual target, or the like, which is presented to the subject eye by the control means controlling the visual target presenting unit. For example, the state of the visual target presenting unit may include the number of times (frequency) of switching the type of the visual target, the number of times (frequency) of switching the visual acuity value of the visual target, and the like. Further, for example, the state of the correction unit may be the state of the correction power of the subject eye, which is obtained by correcting the subject eye by the control means controlling the correction unit. For example, the state of the correction unit may include the number of times (frequency) of switching correction power.

For example, the determination means may determine whether or not the subject eye is being properly examined, as the suitability of the state of the optometry system, based on the reaction time acquired by the reaction time acquisition means. As an example, the determination means may determine that the subject eye is being properly examined by estimating that the examinee see the visual target correctly based on the reaction time. More specifically, for example, in a case where the reaction time is approximately the same as the first threshold value, the state of the visual target presenting unit and the state of the correction unit are appropriate, and thus it may be determined that the examinee can easily recognize the visual target.

Further, as an example, the determination means may determine that the subject eye is not being properly examined by estimating that the examinee is confused about the answer based on the reaction time. More specifically, for example, in a case where the reaction time is longer than the first threshold value, it is estimated that the examinee is confused since the examinee answer slowly, at least one of the state of the visual target presenting unit or the state of the correction unit is inappropriate, and thus it may be determined that the visual target cannot be recognized.

Further, as an example, the determination means may determine that the subject eye has not been properly examined by estimating that the examinee gives a correct answer by chance based on the reaction time. More specifically, for example, in a case where the reaction time is shorter than the first threshold value, it is estimated that the examinee has answered by feeling since the examinee answered quickly, at least one of the state of the visual target presenting unit or the state of the correction unit is inappropriate, and thus it may be determined that the visual target cannot be recognized

### Control Means

The optometry system according to the present embodiment includes control means (for example, the controller 70). The control means controls the operation of the optometry system. For example, the control means may control the operation of the optometry system, based on the start signal for controlling the operation of the optometry system. In this case, the control means may control the operation of the optometry system, based on the start signal automatically input based on the optometry program or the like. Further, in this case, the control means may control the operation of the optometry system, based on a start signal input by the examiner's operation of the operation means (for example, the controller 6). Further, in this case, the control means may control the operation of the optometry system, based on the start signal received by the examiner's operation of the operation means. It is needless to say that, for example, the control means may control the operation of the optometry system, based on a start signal (input signal) input by the operation of the response input unit by the examinee. Further, for example, the control means may control the operation of the optometry system, based on a start signal (input signal) received by the operation of the response input unit by the examinee. For example, an examination (optometry) of a subject eye may be automatically proceeded based on these start signals.

For example, the control means can control at least one of the visual target presenting unit or the correction unit. For example, the control means can control the display of the visual target presenting unit to change at least one of the type of the visual target, the visual acuity value of the visual target, or the like. Further, as an example, the control means can control the correction optical system and change at least one of the correction power that corrects the subject eye, the arrangement of the optical elements, or the like. It is needless to say that, for example, the control means may be enabled to control a means other than the visual target presenting unit and the correction unit.

For example, the control means may change the state of the visual target presenting unit (that is, the state of the visual acuity value of the visual target and the like), based on the suitability of the state of the optometry system. As an example, the control means may change the number of steps for changing the visual acuity value of the visual target, based on the suitability of the state of the optometry system. More specifically, the control means may change the visual acuity value of the visual target by two steps in a case where the state of the optometry system is appropriate, and may change the visual acuity value of the visual target by one step in a case where the state of the optometry system is inappropriate.

Further, for example, the control means may change the state of the correction unit (that is, the state of the correction power and the like), based on the suitability of the state of the optometry system. As an example, the control means may change the number of steps for changing the correction power, based on the suitability of the state of the optometry system. More specifically, the control means may change the correction power by one step (0.25 D) in a case where the state of the optometry system is appropriate, and may change the correction power by two steps (0.5 D) in a case where the state of the optometry system is inappropriate.

For example, the output means may output information including at least reaction time. As an example, the output means may output the reaction time itself In other words, the number of seconds required for the examinee to respond from the start of measurement may be output. Further, as an example, the output means may output the state of the optometry system acquired by the state acquisition means, together with the reaction time. More specifically, along with the reaction time, the state of the visual target presenting unit (at least one state of the type of the visual target, the visual acuity value of the visual target, the number of times of switching the type of the visual target, the number of times of switching the visual acuity value of the visual target, or the like), the state of the correction unit (at least one of the correction power of the subject eye, the number of times of switching the correction power of the subject eye, or the like), and the like may be output. Accordingly, it is easy to determine whether or not the reaction time of the examinee has increased or decreased according to changes in the state of the optometry system.

For example, the output means may output the reaction time and the state of the optometry system, as graph data showing the relationship between the reaction time and the state of the optometry system. As an example, the output means may output graph data showing the relationship between the reaction time and the visual acuity value of the visual target set by the visual target presenting unit. Further, as an example, the output means may output graph data showing the relationship between the reaction time and the correction power set by the correction unit. Accordingly, the change in reaction time of the examinee according to the state of the optometry system can be easily understood.

For example, the leading information output means may repeatedly output the leading information in a case where no signal is obtained from the response input unit even when a predetermined time set for repeating the leading information has elapsed. In this case, the predetermined time may be set to any time by the examiner. In addition, in this case, the predetermined time may be set to a time set in advance based on experiments, simulations, and the like. Further, in this case, the predetermined time may be a time based on a reference value. For example, the time may be the same as the reference value, or the time obtained by increasing or decreasing the reference value by a predetermined amount of time.

For example, at this time, the leading information may always be repeatedly output at predetermined time. That is, the leading information may be repeatedly output at constant time intervals. Further, for example, at this time, the leading information may be repeatedly output at any time. As an example, the output may be repeatedly output at time interval that becomes shorter or longer step by step. Further, as an example, the leading information may be repeatedly output such that the time interval changes according to the number of times of output of the leading information. As a result, the examination can be easily proceeded even when the examinee forget the operation method of the response input unit during the examination.

When repeating the leading information, the leading information output means may change the leading information from the first leading information to the second leading information and output the leading information according to the number of times of output of the leading information. For example, the second leading information may be information at least partially different from the first leading information. As an example, the first leading information may be information representing the operation method for operating the response input unit in order for the examinee to input an answer to the visually recognized examination visual target. Further, as an example, the second leading information may be information representing an operation method for operating the response input unit in order for the examinee to call the examiner. Further, the second leading information may be information including information representing the operation method for the examinee to input an answer and information representing the operation method for calling the examiner. It is needless to say that the first leading information and the second leading information may be information different from these.

As described above, for example, the optometry system according to the present example acquires and outputs the reaction time, in which the reaction time is a time acquired based on a start timing at which measurement of the subject eye is started and a response timing after the start timing, and the reaction time is a time required from a start of the measurement of the subject eye to a response with respect to the measurement of the subject eye, in which the start timing is a timing at which the visual target presenting unit or the correction unit is controlled by the controller, and the response timing is a timing at which a response signal with respect to the measurement is input by the response input unit. Therefore, the examiner can determine whether or not the examination result of the subject eye is accurate from the reaction time of the examinee. Further, the examiner can easily determine the next setting, and the like from the reaction time of the examinee.

Further, for example, the optometry system according to the present example outputs graph data indicating the relationship between the reaction time of the examinee and the optometry system. For example, it is possible to output graph data or the like showing the relationship between the reaction time of the examinee and the correction power set by the correction unit. For example, accordingly, the examiner can easily understand the transition in which the reaction time of the examinee changes according to the state of the optometry system.

In the present example, a configuration in which the correction power for correcting the subject eye E is gradually changed in the refractive correction examination with respective to the subject eye E, has been described as an example, but the present invention is not limited thereto. For example, a configuration may be adopted in which the correction power is changed from a predetermined correction power to a desired correction power each time in the refractive correction examination with respect to the subject eye E. In this case, the control means of the optometry system changes the control state of the correction unit from the initial state to a first correction state, and returns to the initial state after the response in the first correction state is completed. Thereafter, the control means changes the control state of the correction unit from the initial state to a second correction state different from the first correction state. That is, when changing the correction state by the correction unit, the control means once changes the correction state to the initial state, and then changes the correction state to the next correction state.

In addition, in the cross-cylinder examination of the subject eye E, the astigmatism axis angle for correcting the subject eye E is switched in consideration of the correctness of the response of the subject eye E. Therefore, the controller 70 may store the timing (start timing) at which the astigmatism axis angle is switched by controlling the correction optical system 60 and the response timing at which the response of the examinee is obtained. Further, the controller 70 may generate a graph in which the vertical axis is the reaction time of the examinee and the horizontal axis is the astigmatism axis angle for correcting the subject eye E.

1. An optometry system for subjectively measuring an optical characteristic of a subject eye by presenting an examination visual target to the subject eye, the optometry system comprising:
a response input unit by which an examinee reading the examination visual target inputs an answer; and
a controller configured to:
set a reference value based on a reaction time of the examinee with respect to the examination visual target;
automatically proceeds with an examination, based on an input signal from the response input unit; and
output leading information, during the examination of the subject eye, for leading the examinee to input the answer, based on the set reference value.

2. The optometry system according to claim 1,
wherein the controller is further configured to repeatedly outputs the leading information, in a case where no signal from the response input unit is obtained after a predetermined time set for repeating the leading information has elapsed.

3. The optometry system according to claim 2,
wherein the controller is further configured to changes the leading information from first leading information to second leading information that is at least partially different from the first leading information, to output the leading information, according to the number of times of output of the leading information.

4. The optometry system according to claim 1,
wherein the examination includes a plurality of examination items, and
the controller is further configured to set the reference value according to the examination items.

5. The optometry system according to claim 1:
wherein the controller is further configured to:
acquires the reaction time, based on a start timing at which measurement of the subject eye is started and a response timing at which the examinee responds with respect to the examination visual target; and
set the reference value, based on the acquired reaction time.

6. The optometry system according to claim 5,
wherein the controller is further configured to:
acquires the reaction time in real time, during the examination;
changes the reference value from a first reference value to a second reference value different from the first reference value to set the reference value, based on the real-time acquired reaction time; and
output the leading information, based on the second reference value.

7. The optometry system according to claim 5, further comprising:
a visual target presenting unit that emits a target light flux toward the subject eye to present the examination visual target to the subject eye,
wherein the controller is further configured to:
control the visual target presenting unit to automatically proceed with the examination;
acquires a timing at which the controller controls the visual target presenting unit, as the start timing; and
acquires a timing at which the input signal from the response input unit is acquired, as the response timing.

8. The optometry system according to claim 5, further comprising:
a correction unit that changes an optical characteristic of the target light flux to change a correction power for correcting the subject eye,
wherein the controller is further configured to:
controls the correction unit to automatically proceed with the examination; and
acquires a timing at which the controller controls the correction unit, as the start timing.

9. The optometry system according to claim 1, further comprising:
a visual target presenting unit that emits a target light flux toward the subject eye; and
a correction unit that changes an optical characteristic of the target light flux,
wherein the controller is further configured to:
control an operation of the optometry system; and
output the reaction time, and
wherein the reaction time is a time acquired based on a start timing at which measurement of the subject eye is started and a response timing after the start timing, the reaction time being a time required from a start of the measurement of the subject eye to a response with respect to the measurement of the subject eye,
the start timing is a timing at which the visual target presenting unit or the correction unit is controlled by the controller, and
the response timing is a timing at which a response signal with respect to the measurement is input by the response input unit.

10. An optometry system for subjectively measuring an optical characteristic of a subject eye, the optometry system comprising:
a visual target presenting unit that emits a target light flux toward the subject eye;
a correction unit that changes an optical characteristic of the target light flux; and
a controller configured to:
control an operation of the optometry system;
acquires a reaction time; and
output the acquired reaction time,
wherein the reaction time is a time acquired based on a start timing at which measurement of the subject eye is started and a response timing after the start timing, the reaction time being a time required from a start of the measurement of the subject eye to a response with respect to the measurement of the subject eye,
the start timing is a timing at which the visual target presenting unit or the correction unit is controlled by the controller, and
the response timing is a timing at which a response signal with respect to the measurement is input by a response input unit.

11. The optometry system according to claim 10,
wherein the controller is further configured to:
acquire a first reaction time and a second reaction time, that are acquired at different timings, as the reaction time; and
output the first reaction time and the second reaction time in a comparable manner, and
at least one of the start timing or the response timing of the first reaction time and the second reaction time is different.

12. The optometry system according to claim 10,
wherein the controller is further configured to:
control at least one of the visual target presenting unit or the correction unit; and
controls an operation of the optometry system based on the acquired reaction time.

13. The optometry system according to claim 12,
wherein the controller is further configured to:
acquire a state of the optometry system in a case where the visual target presenting unit or the correction means unit is controlled by the controller;
determine suitability of the acquired state of the optometry system, based on the acquired reaction time; and
control an operation of the optometry system, based on a determined suitability.

14. The optometry system according to claim 13,
wherein the controller is further configured to
output the acquired state of the optometry system, together with the acquired reaction time.

15. The optometry system according to claim 14,
wherein the controller is further configured to
output data indicating a relationship between the reaction time and the state of the optometry system.

16. The optometry system according to claim 15,
wherein the controller is further configured to
output graph data indicating a relationship between the reaction time and the state of the optometry system.

17. The optometry system according to claim 16,
wherein the controller is further configured to
output graph data indicating a relationship between the reaction time and a correction power set by the correction unit.

18. A non-transitory computer-readable recording medium storing an optometry program used in an optometry system for subjectively measuring an optical characteristic of a subject eye by presenting an examination visual target to the subject eye, the optometry program being executed by a processor to cause the optometry system to perform:
a setting processing of setting a reference value based on a reaction time of an examinee with respect to the examination visual target;
an answering processing in which the examinee reading the examination visual target inputs an answer;
a control processing of automatically proceeding with an examination, based on an input signal of the answering processing; and
a leading information output processing of outputting leading information, during the examination of the subject eye, for leading the examinee to input the answer, based on the reference value set in the setting processing.

19. (canceled)
